SEQUENCE LISTING (5 sequences)

SEQ ID NO: 1 - DNA, 29 bp, Artificial
  Primer
agcgaattcatgtctgagcaaggagaact

SEQ ID NO: 2 - DNA, 30 bp, Artificial
  Primer
acgggatccggagagtctgatcctggtgac

SEQ ID NO: 3 - DNA, 33 bp, Artificial
  Primer
acctgctcctgacttggcttttcttctctcttg

SEQ ID NO: 4 - DNA, 33 bp, Artificial
  Primer
caagagagaagaaaagacaagtcaggagcaggt

SEQ ID NO: 5 - DNA, 33 bp, Artificial
  Primer
acctgctcctgacttgtcttttcttctctcttg

(19) United States
(12) Patent Application Publication
(10) Pub. No.: US 2005/0153372 A1
(43) Pub. Date: Jul. 14, 2005

(54) Compositions and method for regulation of calcium-dependent signaling in brain

(76) Inventors: Paul Greengard, New York, NY (US); Sergey Rakhilin, Yorktown, NY (US); Angus Nairn, New York, NY (US)

Correspondence Address: JONES DAY, 222 EAST 41ST ST, NEW YORK, NY, 10017, US

(21) Appl. No.: 10/505,537

(22) PCT Filed: Feb. 21, 2003
(86) PCT No.: PCT/US03/05273

Related U.S. Application Data
(60) Provisional application No. 60/358,548, filed on Feb. 21, 2002.

(51) Int. Cl. G01N 33/53; G01N 33/542

(52) U.S. Cl. 435/7.9

(57) ABSTRACT

Methods are provided for screening agents for activity as modulators of calcium signaling in brain tissue.

This application claims the benefit, under 35 U.S.C. § 119(e), to U.S. provisional application no. 60/358,548, filed Feb. 21, 2002, which is incorporated herein by reference in its entirety.

### 1. TECHNICAL FIELD

The invention provides, first, methods for screening agents for activity to modulate calcium signaling in the nervous system involving DARPP-21. For example, the present invention provides methods for screening agents that inhibit or augment protein-protein binding between DARRP-21 and calmodulin, and methods for screening candidate calmodulin-binding proteins whose binding is inhibited or augmented by DARPP-2 1. The present invention also relates to compositions and methods for modulating DARPP-21-related signaling pathways, e.g., dopaminergic signaling pathways. For example, the present invention relates to methods and compositions for modulating the phosphorylation state, protein expression levels and/or protein-protein interactions of DARPP-21 in such signaling pathways. The present invention also relates to compositions and methods of preventing, treating, or ameliorating the symptoms of a dopamine-related disorder, e.g., by administering an agent that modulates formation of DARPP-21/calmodulin complexes.

### 2. BACKGROUND OF THE INVENTION

Elevations of intracellular calcium levels are ubiquitous to all cells as a response to a variety of stimuli such as neurotransmitters, hormones, drugs and cellular depolarization. Neuronal calcium-dependent signaling is involved in a wide range of physiological and pathological processes as important and diverse as memory acquisition, epilepsy and brain ischemia.

In certain cells of the central nervous system (CNS), such as those in cellular networks utilizing the neurotransmitter dopamine, the cAMP-dependent signaling pathway is activated in response dopamine, serotonin, opiates and other agents. (Greengard (2000) Science 294:1024-1029). A number of disorders are closely associated with this dopaminergic cellular network of the CNS such as Parkinson's disease, attention deficit hyperactivity disorder (ADHD), schizophrenia, and drug abuse. One functional output of the cAMP-dependent signaling pathway in the dopaminergic cellular network is the modulation of the calcium-signaling pathway, although the molecular mechanisms of the interaction between the calcium-signaling pathway and cAMP-dependent signaling pathway are not clear. Thus, there is a critical need in the art to provide suitable methods for elucidating agents that regulate the inter-relationship between the calcium-dependent and cAMP-dependent signaling pathways that may serve as candidates for therapeutic use.

DARPP-21 is a neuronal 21 kDa dopamine- and cAMP-regulated phosphoprotein that is highly expressed in dopamine-enriched regions of the brain. DARPP-21 contains no conserved domains and has displayed no similarity to any known protein until the recent discovery of a 100 kDa protein in thymus that contained the entire DARPP-21 as a part of its N-terminal sequence (Kiselow, J. et al. 2001. Eur. J. Immunol. 4:1141-1149). This protein, known as TARPP (thymus-specific cAMP-regulated phosphoprotein), has not been detected in brain and its function remains unknown.

Sequencing and biochemical analysis of DARPP-21 have shown that it is phosphorylated on Ser⁵⁵ by the cAMP-dependent protein kinase (also known as protein kinase A or PKA; Williams, K. R. et al. 1989. J. Neurosci. 9:3631-3637). Immunohistochemical analysis in rat brain demonstrated that DARPP-21 is enriched in the basal ganglia, with the highest levels of immunoreactivity seen in structures comprising the limbic striatum (Ouimet, C. C. et al. 1989. J Neurosci. 9:865-875). These areas of mammalian brain are known to be densely populated by dopaminergic neurons. Using phosphorylation state-specific antibodies selective for detection of DARPP-21 phosphorylated on Ser⁵⁵, it was found that activation of D1 dopamine receptors increased the level of DARPP-21 phosphorylation in striatal slices due to up-regulation of PKA activity. Conversely, activation of D2 dopamine receptors caused a decrease in DARPP-21 phosphorylation (Caporaso, G. L. et al. 2000. Neuropharmacology 39:1637-1644). In addition, treatment of mice with methamphetamine or cocaine resulted in increased DARPP-21 phosphorylation in vivo. Protein phosphatase-2A was shown to be primarily responsible for dephosphorylation of DARPP-21 in mouse striatum (Caporaso, G. L. et al. 2000. Neuropharmacology 39:1637-1644).

These data indicate that DARPP-21 is effected by, and perhaps mediates, physiologic effects of dopamine and certain drugs of abuse in the dopaminergic cellular network in the CNS. It is unclear what, if any, role DARPP-21 might have in these processes, and, in particular, what, if any role DARPP-21 might have in effecting calcium-dependent and/or cAMP-dependent signaling pathways.

### 3. SUMMARY OF THE INVENTION

It has now been found that DARPP-21 phosphorylation modulates calcium-dependent signaling in the central nervous system. In particular, the present invention is based, in part, on the surprising discovery, on the part of the inventors, that the phosphorylation state of DARPP-21 regulates its interaction with calcium/calmodulin under physiological conditions. It is noted that DARPP-21 was previously referred to as ARPP-21; see, e.g., for example, U.S. Provisional Patent No. 60/358,548.

An object of the present invention is a method for screening agents for an ability to modulate dopaminergic signaling pathways, in particular, DARPP-2 1-related dopaminergic signaling pathways. In one embodiment, such methods relate to screening agents for an ability to modulate calcium signaling in cells or tissue.

In one non-limiting example, such a method can comprise determining a first level of phosphorylation of DARPP-21 in a first or control sample of cells or tissue; contacting a second sample of cells or tissue with an agent to be tested for the ability to regulate calcium signaling; determining a second level of phosphorylation of DARPP-21 in said cells or tissue; and comparing said first and second levels of phosphorylation, wherein a difference in said levels is indicative of the ability of the agent to modulate DARPP-21 phosphorylation and thus modulate calcium signaling. In one embodiment, the cells or tissues are brain cells or tissue. Such methods can further comprise testing whether the agent modulates a biological activity of, e.g., a calmodulin-dependent protein such as an enzyme, for example a phosphodiesterase, a kinase, a phosphatase or an adenylyl cyclase, calmodulin-dependent kinase I (CaMKI) and/or protein phosphatase 2B (calcineurin).

In another non-limiting example, such a method can comprise determining whether an agent binds DARPP-21. For example, an agent can be contacted with DARPP-21 for a time sufficient to allow binding, DARPP-21 can be washed to remove unbound agent, and the presence of agent bound to DARPP-21 can be assayed so that if the agent binds DARPP-21 then an agent to be tested for an ability to modulate calcium signaling is identified.

In another non-limiting example, such a method can comprise use of a protein-protein binding assay which includes DARPP-21 and calmodulin in the presence of an agent, and determining whether the agent inhibits or augments the protein-protein binding between DARPP-21 and calmodulin, wherein inhibition or augmentation of the protein-protein binding is indicative of the agent to modulate intracellular calcium signaling. Such a method can, for example, further comprise first testing whether the agent binds DARPP-21 and/or binds calmodulin so that if the agent binds DARPP-21 and/or calmodulin, the agent is then tested in the protein-protein binding assay. Such methods can also, or in the alternative, further comprise testing whether the agent modulates a biological activity of, e.g., calmodulin-dependent kinase I (CaMKI) and/or protein phosphatase 2B (calcineurin). In one embodiment, DARPP-21 comprises a negative charge at amino acid position 55. In another embodiment, DARPP-21 is not phosphorylated. In yet another embodiment, DARPP-21 is phosphorylated.

In yet another non-limiting example of such a method, is a method of screening candidate DARPP-21-binding peptides, whose binding with DARPP-21 is inhibited or augmented by calmodulin comprising performing a protein-protein binding assay which includes DARPP-21 and a candidate DARPP-21-binding peptide in the presence of calmodulin, and determining whether calmodulin inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In yet another non-limiting example of such a method, is a method of screening candidate calmodulin-binding peptides, whose binding with calmodulin is inhibited or augmented by DARPP-21 comprising performing a protein-protein binding assay which includes calmodulin and a candidate calmodulin-binding peptide in the presence DARPP-21, and determining whether DARPP-21 inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In another non-limiting example of such a method, is a method of screening candidate DARPP-21-binding agents, e.g., peptides, whose binding with DARPP-21 is inhibited or augmented by calmodulin comprising performing a protein-protein binding assay which includes DARPP-21 and a candidate peptide in the presence calmodulin, and determining whether calmodulin inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. In yet another embodiment, Ser⁵⁵ in DARRP-21 is mutated by its replacement with a another amino acid. Such methods can further comprise testing whether the agent modulates a biological activity of, e.g., a calmodulin-dependent protein such as an enzyme, for example a phosphodiesterase, a kinase, a phosphatase or an adenylyl cyclase.

In yet another non-limiting example of such a method, is a method of screening candidate calmodulin-binding agents, e.g., peptides, whose binding with calmodulin is inhibited or augmented by DARPP-21 comprising performing a protein-protein binding assay which includes calmodulin and a candidate polypeptide in the presence DARPP-2 1, and determining whether DARPP-21 inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

A further object of the invention is methods and compositions for modulating a biological activity (e.g., the phosphorylation state, protein expression levels and/or protein-protein interactions) of DARPP-21 in dopamine signaling pathways. For example, the present invention presents methods and compositions for modulating calcium signaling, e.g., neuronal calcium signaling, in such pathways. In one embodiment, such methods can comprise contacting a cell or tissue with an agent that modulates a biological activity of DARPP-21, such that the signaling pathway is modulated. The contacting agent can, for example, be one that alters the phosphorylation state of DARPP-21. The contacting agent can, for example, be one that alters formation of DARPP-21/calmodulin complexes.

Yet another object of the present invention is a method for preventing, treating, or ameliorating the symptoms of a calcium, e.g., dopamine-related disorder by administering to a subject in need thereof an agent that modulates a biological activity of DARPP-21. In one embodiment, the agent administered alters the phosphorylation state of DARPP-21. In another embodiment, the agent administered alters formation of DARPP-21/calmodulin complexes.

### 4. DESCRIPTION OF THE FIGURES

FIG. 1 illustrates representative results of a yeast two-hybrid protein-protein binding assay indicating that DARPP-21 interacts with calmodulin in yeast using a galactose/glucose test.

FIG. 2 illustrates representative results of a yeast two-hybrid protein-protein binding assay indicating that only phospho-Ser⁵⁵-DARPP-21 binds calmodulin since mutation of Ser⁵⁵ to alanine in DARPP-21 abrogates calmodulin and DARPP-21 binding.

FIG. 3 illustrates representative results of a coimmunoprecipitation protein-protein binding assay in which polyclonal anti-DARPP-21 antibody coimmuniprecipitates calmodulin together with DARPP-21 from striatal extracts prepared from bovine brain.

FIG. 4 illustrates representative results of a immobilized protein-protein binding assay involving DARPP-21 affinity chromatography indicating the specific binding between calmodulin and DARPP-21 in an immobilized DARPP-21 column (+lanes) but not control column (−lanes).

FIG. 5 illustrates representative results of a immobilized protein-protein binding assay involving calmodulin affinity chromatography indicating the specific binding between calmodulin and DARPP-21 in the presence, but not absence, of calcium.

FIG. 6 illustrates the selective inhibition of CaMKI by phosphorylated DARPP-21 but not by un-phosphorylated DARPP-21.

FIG. 7 illustrates the selective inhibition of calcineurin by phosphorylated DARPP-21 but not by un-phosphorylated DARPP-21.

### 5. DETAILED DESCRIPTION OF THE INVENTION

The present invention is based, in part, on the surprising discovery, on the part of the inventors, that the phosphorylation state of DARPP-21 regulates its interaction with calcium/calmodulin under physiological conditions and can be utilized in methods for screening agents for the regulation of the calcium-dependent signaling. On the basis of these findings, the formation of DARPP-21/calmodulin complexes are identified as a target for agents capable of modulating intracellular calcium signaling, of modulating neuronal cell calcium signaling and of preventing, treating, or ameliorating the symptoms of a dopamine-related disorder.

Without intending to be bound by any particular theory, it is believed that phosphorylation of DARPP-21 increases its affinity for calmodulin and thereby competitively inhibits components of the calcium-signaling pathway, including for example, calmodulin-dependent kinase I (CaMKI and protein phosphatase 2B (calcineurin). Because DARPP-21 phosphorylation is attendant with dopamine stimulation of PKA, therefore, dopamine stimulation of PKA is linked to a calcium-signaling mechanism. In this pathway, activation of D1 receptors stimulates cAMP synthesis by adenylyl cyclase, which in turn activates PKA and results in phosphorylation of DARPP-21, among other proteins. Thus, the present invention describes a phosphorylation-dependent mechanism that regulates calcium signaling within neurons.

An object of the present invention is a method for screening agents for an ability to modulate dopaminergic signaling pathways, in particular, DARPP-21-related dopaminergic signaling pathways. In one embodiment, such methods relate to screening agents for an ability to modulate calcium signaling in cells or tissue.

One aspect of the invention provides a method for screening for novel therapeutics that regulate neuronal calcium-dependent signaling by affecting DARPP-21 phosphorylation in brain tissues, wherein regulation of phosphorylation of DARPP-21 leads to changes in calcium-dependent signaling The method involves identifying agents capable of modulating phosphorylation of DARPP-21 in cells or tissues either in vivo or in vitro. In the context of the present invention, “modulate” is defined as either an increase or a decrease in phosphorylation when used with respect to DARPP-21 phosphorylation. The method can be based on the determination of phosphorylation levels of DARPP-21 both before and after treatment of cells or tissues with the agent to be tested, or, alternatively, can be based on determination of phosphorylation levels of DARPP-21 after treatment of cells or tissue with the agent to be tested, and comparing the levels to a control or a known standard.

In one non-limiting example, such a method can comprise determining a first level of phosphorylation of DARPP-21 in a first or control sample of cells or tissue; contacting a second sample of cells or tissue with an agent to be tested for the ability to regulate calcium signaling; determining a second level of phosphorylation of DARPP-21 in said cells or tissue; and comparing said first and second levels of phosphorylation, wherein a difference in said levels is indicative of the ability of the agent to modulate DARPP-21 phosphorylation and thus modulate calcium signaling. In one embodiment, the cells or tissues are brain cells or tissue. Such methods can further comprise testing whether the agent modulates a biological activity of, e.g., a calmodulin-dependent protein such as an enzyme, for example a phosphodiesterase, a kinase, a phosphatase or an adenylyl cyclase.

In certain embodiments of the method, the cells or tissues are brain cells or tissue. The in vitro and in vivo applications would include but not be limited to modulating activity in whole animals, in tissue slices, in broken cell preparations, in intact cells, and in isolated and purified cell preparations. A cell or tissue may include, but not be limited to: an excitable cell, e.g., a sensory neuron, motoneuron, or interneuron; a glial cell; a primary culture of cells, e.g., a primary culture of neuronal or glial cells; cell(s) derived from a neuronal or glial cell line; dissociated cell(s); whole cell(s) or intact cell(s); permeabilized cell(s); a broken cell preparation; an isolated and/or purified cell preparation; a cellular extract or purified enzyme preparation; a tissue or organ, e.g. brain, brain structure, brain slice, spinal cord, spinal cord slice, central nervous system, peripheral nervous system, or nerve; tissue slices, and a whole animal. In certain embodiments, the brain structure is striatum, cerebral cortex, the hippocampus, or their anatomical and/or functional counterparts in other mammalian species. In certain embodiments, the cell or tissue is a primary neuronal culture, a hippocampal tissue explant or reaggregate cultures from fetal brain.

The phosphorylation levels of DARPP-21 can be assessed using techniques known to those of skill in the art. These techniques may include, for example, the use of radioisotopes or phosphorylation state-specific antibodies, or the analysis of the chromatographic or electrophoretic mobility of DARPP-21 or a polypeptide fragment produced from DARPP-21, or the detection of phosphoserine from hydrolyzed DARPP-21, or any other suitable technique either alone or in any combination.

In one embodiment of the present invention, the modulation results in increased levels of DARPP-21 phosphorylation. In another embodiment, the modulation results in decreased levels of DARPP-21 phosphorylation.

In another non-limiting example, such a method can comprise determining that an agent that binds DARPP-21. For example, an agent can be contacted with DARPP-21 for a time sufficient to allow binding, the DARPP-21 can be washed to remove unbound agent and the presence of DARPP-21 agent can be assayed so that if the agent binds DARPP-21 then an agent to be tested for an ability to modulate calcium signaling is identified.

Another category of screening agents of the present invention involves “protein-protein binding assays”, which as used herein, refer to any assay in which two non-identical proteins are determined either to bind or not bind to each other, wherein at least one of the proteins is calmodulin or DARPP-21. Such an assay may be conducted in the absence or in the presence of the agent to be screened. In one embodiment, a protein-protein binding assay may utilize isolated, purified and/or recombinantly expressed forms of the calmodulin or DARPP-21 assay components. Isolated or purified polypeptides of the invention are those which have been separated from their native environment (e.g., cytoplasmic fraction from a cell or by recombinant procedure). Recombinantly expressed polypeptides can be expressed and obtained using standard techniques well known in the art. In another embodiment, a protein-protein binding assay is performed using non-isolated or non-purified proteins.

In another non-limiting example, such a method can comprise use of a protein-protein binding assay which includes DARPP-21 and calmodulin in the presence of an agent, and determining whether the agent inhibits or augments the protein-protein binding between DARPP-21 and calmodulin, wherein inhibition or augmentation of the protein-protein binding is indicative of the agent to modulate intracellular calcium signaling. Such a method can, for example, further comprise first testing whether the agent binds DARPP-21 and/or binds calmodulin so that if the agent binds DARPP-21 and/or calmodulin, the agent is then tested in the protein-protein binding assay. Such methods can also, or in the alternative, further comprise testing whether the agent modulates a biological activity of, e.g., calmodulin-dependent kinase I (CaMKI) and/or protein phosphatase 2B (calcineurin). In one embodiment, DARPP-21 comprises a negative charge at amino acid position 55. In another embodiment, DARPP-21 is not phosphorylated. In yet another embodiment, DARPP-21 is phosphorylated.

In yet another non-limiting example of such a method, is a method of screening candidate DARPP-21-binding peptides, whose binding with DARPP-21 is inhibited or augmented by calmodulin comprising performing a protein-protein binding assay which includes DARPP-21 and a candidate DARPP-21-binding peptide in the presence calmodulin, and determining whether calmodulin inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In yet another non-limiting example of such a method, is a method of screening, candidate calmodulin-binding peptides, whose binding with calmodulin is inhibited or augmented by DARPP-21 comprising performing a protein-protein binding assay which includes calmodulin and a candidate calmodulin-binding peptide in the presence DARPP-21, and determining whether DARPP-21 inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In another non-limiting example of such a method, is a method of screening candidate DARPP-21-binding agents, e.g., peptides, whose binding with DARPP-21 is inhibited or augmented by calmodulin comprising performing a protein-protein binding assay which includes DARPP-21 and a candidate peptide in the presence calmodulin, and determining whether calmodulin inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In yet another non-limiting example of such a method, is a method of screening candidate calmodulin-binding agents, e.g., peptides, whose binding with calmodulin is inhibited or augmented by DARPP-21 comprising performing a protein-protein binding assay which includes calmodulin and a candidate polypeptide in the presence DARPP-21, and determining whether DARPP-21 inhibits or augments the protein-protein binding. In one embodiment, DARPP-21 comprises a negative charge at amino acid position 55. In another embodiment, DARPP-21 is not phosphorylated. In yet another embodiment, DARPP-21 is phosphorylated. Such methods can further comprise testing whether the agent modulates a biological activity of, e.g., a calmodulin-dependent protein such as an enzyme, for example a phosphodiesterase, a kinase, a phosphatase or an adenylyl cyclase.

In one embodiment, DARPP-21 comprises a negative charge at amino acid position 55. “Amino acid position 55” refers to the position that corresponds to the PKA phosphorylation site (typically Ser⁵⁵) in wildtype DARPP-21 regardless of whether that site has been mutated to a residue that cannot be phosphorylated by PKA. In another embodiment, DARPP-21 is not phosphorylated. In yet another embodiment, DARPP-21 is phosphorylated.

In a certain embodiment, DARPP-21 comprises a negative charge at amino acid 55. In another embodiment DARPP-21 is not phosphorylated. In yet another embodiment DARRP-21 is phosphorylated.

One of skill in the art knows that protein-protein binding assay may be performed in vivo, in vitro, or in situ. Methods for performing protein-protein binding assays are known by one of skill in the art. It is intended that any method suitable for determining whether two proteins bind together are encompassed within the meaning of protein-protein binding assay. Thus, for example, a protein-protein binding assay may involve immobilizing one protein to a suitable support and contacting it with a second non-immobilized protein (“immobilized protein-protein binding assay”). Such a support may be porous or non-porous and, for example, could be a resin, bead, membrane such as PVDF, nylon66, nitrocellulose, plastic etc., microtiter plate well, dipstick, and the like without limitation. Two examples of immobilized protein-protein binding assays using affinity chromatography are presented in Section 6.4.3 below. Other exemplary protein-protein binding assays include, without intending to limit the possibilities, the use of cofractionation, coimmunoprecipitation (see Section 6.4.2 for an example), overlay blotting (e.g., Murray et al. 2001 Biotechniques 30:1036-42), gel exclusion, chromatofocusing, sedimentation, yeast two hybrid system (see Section 6.4.1 for an example), yeast three hybrid system (Licitra & Liu 1996 Proc. Natl. Acad. Sci. (USA) 93:12817-12821), E. coli/BCCP interactive screening system (for a general description and references, see Gemrino et al. 1993 Proc. Natl. Acad Sci. (U.S.A) 90: 1639, incorporated herein by reference in its entirety), and so forth.

In addition, the present invention provides methods for designing new chemical agents that have activity as modulators of calcium-dependent signaling pathways, where these new chemical agents may include, but not be limited to, any agent with the ability to either stimulate or inhibit calcium-dependent signaling through its ability to affect phosphorylation of DARPP-21. Also contemplated in the present invention are agents that may prevent the interaction of phosphorylated DARPP-21 with calmodulin, agents that would include but not be limited to peptides and small organic molecules. Further, the present invention includes agents and use of agents that have the ability to mimic phosphorylated DARPP-21 activity in cells and tissues and therefore also affect calcium-dependent signaling through their ability to mimic DARPP-21.

The present invention also includes compositions identified by the methods of the present invention. One of skill would understand that once identified as capable of modulating activity in any one of the methods of the present invention, the agent can, for example, be further tested for an ability of the agent to be used therapeutically to modulate calcium-dependent signaling in neuronal cells in order to treat conditions in which this signaling may be involved. Therefore, also included in the present invention are compositions identified by the screening methods that can be used in the treatment of diseases related to calcium-dependent signaling such as epilepsy, schizophrenia, Parkinson's disease, attention deficit hyperactivity disorder, depression, drug abuse, pain, cancer, stroke, Alzheimer's disease, Huntington's disorder or Tourette's syndrome. A preferable embodiment is a composition for preventing, treating, or ameliorating the symptoms of a dopamine-related disorder wherein the composition alters formation of DARPP-21/calmodulin complexes.

A further object of the invention is methods and compositions for modulating a biological activity (e.g., the phosphorylation state, protein expression levels and/or protein-protein interactions) of DARPP-21 in dopamine signaling pathways. For example, the present invention presents methods and compositions for modulating calcium signaling, e.g., neuronal calcium signaling, in such pathways. In one embodiment, such methods can comprise contacting a cell or tissue with an agent that modulates a biological activity of DARPP-21, such that the signaling pathway is modulated. The contacting agent can, for example, be one that alters the phosphorylation state of DARPP-21. The contacting agent can, for example, be one that alters formation of DARPP-21/calmodulin complexes. The contacting agent can, for example, be one that alters the ability of DARPP-21 to inhibit the activity of a calcium/calmodulin-dependent enzyme.

Yet another object of the present invention is a method for preventing, treating, or ameliorating the symptoms of a dopamine-related disorder by administering to a subject in need thereof an agent that modulates a biological activity of DARPP-21. In one embodiment, the agent administered alters the phosphorylation state of DARPP-21. In another embodiment, the agent administered alters formation of DARPP-21/calmodulin complexes. Among the dopamine-related disorders that can be modulated are, for example, such disorders as epilepsy, schizophrenia, Parkinson's disease, attention deficit disorder, depression, drug abuse, pain, cancer, stroke, and Alzheimer's disease.

Any agent identified via the methods described herein can be utilized as part of the modulation and/or treatment-related methods of the present invention. Further, any agent that modulates a biological activity of DARRP-21 can be utilized as part of these methods. Among the agents that can be utilized as part of the modulation and/or treatment-related methods of the invention are agents that effect the phosphorylation state of DARRP-21, either directly or indirectly. For example, agents that activate or inhibit PKA can also affect DARPP-21 phosphorylation and, as a result, influence calcium signaling. As such, for example, agonists or antagonists of the dopamine D1 and/or D2 receptors can be utilized.

Moreover, serotonin affects PKA activity via stimulation of serotonin receptors. In certain embodiments the agent utilized, therefore, acts a ligand for a serotonin receptor, e.g. 5-HT4 or 5-HT6. For example, an agent can represent a selective serotonin reuptake inhibitor (SSRI).

Glutaminergic receptor activation is known to lead to PKA down-regulation. For example, metabotropic glutamate receptors type 1 (mGluR1) stimulate casein kinase I (CK1) which, in turn, activates cdk5. The latter kinase phosphorylates DARPP-32 at Thr⁷⁵ Phospho-DARPP-32 inhibits PKA and promotes de-phosphorylation of the DARPP-21. Hence, among the agents that can be utilized are glutamate receptor ligands, casein kinase 1, cdk5, or DARPP-32, or agonists or antagonist therefor. In another embodiment the composition is cholecystokinin which stimulates release of glutamate, or an agonist or antagonist thereof.

Additional points of the regulatory pathway leading to PKA activation includes certain G-proteins and adenylyl cyclase. Thus, agents that regulate these protein families, for example, forskolin and pertussis toxin can be utilized as agents in the methods described herein. The second messenger cAMP which activates PKA, along with its phosphodiesterase-resistant analogs, such as 8-bromo-cAMP for example, represent additional compositions that can impact calcium-signaling through DARPP-21. Other important proteins, among their modulators, that impact phosphorylation levels of PKA substrates include phosphodiesterases, and kinases including PKA and its inhibitors such as PKL Rp-cAMPS, the PKA regulatory subunit, phospho-Thr⁷⁵-DARPP-32, and H89, as well as phosphatases (e.g., PP1 and PP2A) and their activators or inhibitors such as okadaic acid, calyculin, and cantharidin and analogues of these phosphatase inhibitors.

Agents that directly affect calmodulin binding, preferably those that inhibit or augment calmodulin binding to DARPP-21, can also be utilized as part of the methods described herein. Components of the calcium-signaling pathway that are impacted by the phosphorylation of DARPP-21, or agonists or antagonist therefor, can also be utilized as part of the methods described herein. For example, phospho-DARPP-21 is a competitive inhibitor of calcineurin, and calcineurin substrates include endocytotic-related and cytoskeletal-associated proteins such as dynamin and tau factor, channel/receptor proteins including the NMDA receptor, kinases such as the type II regulatory subunit of PKA, kinase activity modulators including DARPP-32, enzymes such as NO synthase, and transcription factors such as Elk-1 and NFAT4. DARPP-21 also inhibits CaMKI, as described in Section 6.5, and CaMKI itself phosphorylates substrates such as CaMKK, proteins associated with synaptic vesicle release such as synapsin I and II, and transcription factors including CREB and ATF-1. As such, any of these compounds, or agonists or antagonist therefor, can be utilized as agents as part of the methods described herein.

The following non-limiting examples are presented to further illustrate the present invention.

### 6. ILLUSTRATIVE EXAMPLES

The following examples are meant to illustrate the principles and advantages of the present invention. They are not intended to be limiting in any way.

### 6.1 Example 1

### Protein Phoshorylation Reactions

For preparations of phosphorylated DARPP-21, the protein was phosphorylated in vitro using [γ-³²P]ATP with the catalytic subunit of PKA as described (Caporaso et al. 2000. Neuropharmacology 39:1636-1643). The stoichiometry of phosphorylation was determined to be 95% for DARPP-2 1.

### 6.2 Example 2

### Immunostaining

Immunostaining was employed to detect either calmodulin or DARPP-21 in samples as follows. Western blot analysis of calmodulin was performed with minor modifications as described by Van Eldik and Wolchok (1984. BBRC 124:752-759). Proteins were separated by SDS-PAGE and electrophoretically transferred to PVDF membrane for 20 minutes (calmodulin) or 60 minutes (DARPP-21). The membrane was then rinsed twice with phosphate-buffered saline (PBS) and fixed with 0.2% glutaraldehyde/PBS for 45 minutes at room temperature. The membrane was then blocked with 5% dry milk solution in PBS/0.05% Tween-20 for 1 hour followed by incubation with primary antibodies. DARPP-21 was identified using rabbit polyclonal #204 or monoclonal 6A antibodies. Anti-calmodulin monoclonal antibody was obtained from Upstate Biotechnology, Inc. (Lake Placid, N.Y.).

### 6.3 Example 3

### Construction and Expression of Fusion Proteins

For construction and expression of fusion proteins, DARPP-21 forward (5′-AGCGAATTCATGTCTGAGCAAGGAGAACT-3′; SEQ ID NO:1) and DARPP-21 reverse (5′-ACGGGATCCGGAGAGTCTGATCCTGGTGAC-3′; SEQ ID NO:2) primers were used to amplify DARPP-21 cDNA and generate the DARPP-21/LexA fusion protein in yeast. Ala forward (5′-ACCTGCTCCTGACTTGGCTTTTCTTCTCTTCTTG-3∝; SEQ ID NO:3), Asp forward (5′CAAGAGAGAAGAAAAGACAAGTCAGGAGCAGGT-3′; SEQ ID NO:4) and Asp reverse (5′-ACCTGCTCCTGACTTGTCTTTTCTTCTCTCTTG-3′; SEQ ID NO:5) primers were used to generate DARPP-21 mutants with Ala or Asp, respectively, substitutions for Ser⁵⁵. Obtained mutations were confirmed by DNA sequencing. DARPP-21 C-terminal 6×His tag fusion protein was generated, expressed and purified as described by Caporaso et al. (2000. Neuropharmacology 39:1636-1643).

### 6.4 Example 4

### Protein-Protein Binding Assays to Screen for Protein-Protein Interaction Involving Calmodulin and DARPP-21

### 6.4.1 Yeast Two-Hybrid Protein-Protein Binding Assay

Protein-protein binding assays were performed in a yeast two-hybrid system using the DupLexA system (Origene Technologies, Rockville, Md.) in yeast strain EGY48. To generate a fusion protein of LexA and DARPP-21, DARPP-21 cDNA was subcloned into EcoRI- XhoI sites of pEG202 bait vector. Prey vector contained oligo-dT primed rat brain library (Origene Technologies, Rockville, Md.) attached to the B42 acid blob domain and governed by a gal-1 promoter.

Yeast were sequentially transformed with the bait, pSH 18-34 lacz-harboring prey cDNA library-containing plasmids using the lithium acetate protocol. Efficiency of transformation was estimated to be 10⁶ with a library complexity of 4×10⁶ individual clones. Positive clones appeared 3 to 7 days after transformation. Bait-prey interaction was assessed by intensity of growth on a (gal)(-his, -trp, -ura, -leu) medium. Specificity of the interaction was verified by X-gal staining and mating assay (Origene Technologies, Rockville, Md.).

To confirm calmodulin-DARPP-21 interactions and compare calmodulin binding to DARPP-21 mutants, EGY48 yeast cells were transformed with the bait, prey and lacZ reporter plasmids and plated onto (glu)(-his, -trp, -ura) plates. Four days later, selected colonies were transferred into a liquid (+raf)(-his, -trp, -ura) medium and grown overnight at 30° C. with vigorous shaking. A series of 10-fold dilution of the cultures was prepared and spotted (10 microliters) onto (glu)(-his, -trp, -ura) (positive control), (glu)(-his, -trp, -ura, -leu) (negative control), and (gal)(-his, -trp, -ura, -leu) test plates.

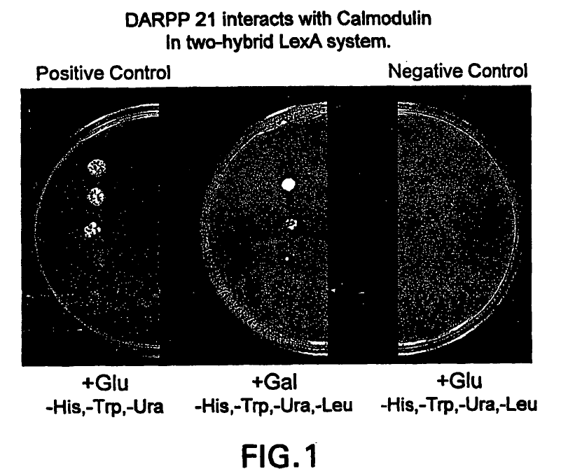

A representative result of an initial protein-protein binding assay, was that a 1500 base pair (bp) cDNA was retrieved that contained the full-length calmodulin sequence. The DARPP-21-calmodulin interaction in yeast was verified using galactose/glucose test (FIG. 1), white/blue staining of the obtained colonies, and the mating test.

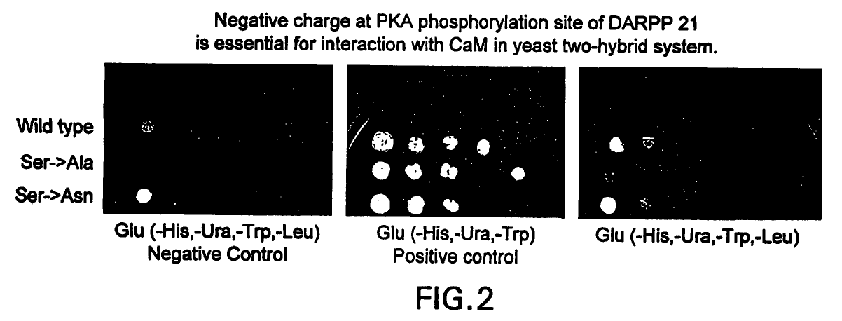

Since DARPP-21 is a PKA substrate, experiments were performed utilizing the yeast two-hybrid protein-protein binding assay to examine the relationship between state of phosphorylation of the DARPP-21 and its binding to calmodulin binding. Mutation of Ser⁵⁵ to alanine in DARPP-21 completely inhibited the interaction with calmodulin (FIG. 2). However, substitution of serine with negatively charged aspartic acid did not have such an effect. These data indicated that in yeast, calmodulin predominantly, if not exclusively, interacts with phosphorylated forms of DARPP-21.

### 6.4.2 Coimmunoprecipitation Protein-Protein Binding Assay

Bovine striatum (2 grams) was homogenized in 10 ml of buffer A which contained 150 NaCl, 20 mM Tris-HCl, pH 7.5, 0.5 mM CaCl₂, 1% Triton X-100, and EDTA-free protease inhibitor cocktail (Roche Diagnostics Corporation, Indianapolis, Ind.). Insoluble material was removed by brief centrifugation at 100,000× g for 15 minutes. The supernatant was diluted with buffer A to a final protein content of 1 mg/ml. The extract (1 ml) was incubated with anti-calmodulin or anti-DARPP-21 antibodies overnight at 4° C. Protein A-Sepharose slurry (70 μl of 50% w/v; Amersham Pharmacia Biotech, Piscataway, N.J.) was added to each sample and the samples further incubated for 3 hours. The beads were washed 3 times with modified buffer A containing 300 mM NaCl and bound proteins were eluted with SDS-PAGE sample buffer. Obtained samples were fractionated on a 4-20% gradient SDS Tris/glycine gel (Novex) and analyzed by immunostaining as described in Section 6.2 above.

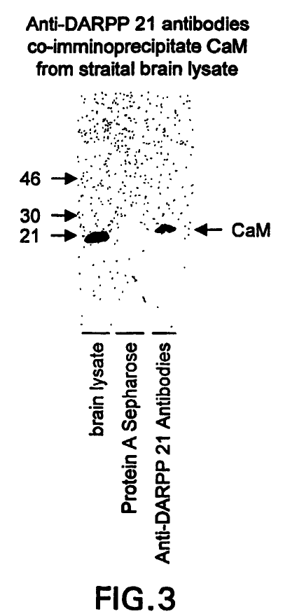

Representative results as shown in FIG. 3 illustrate the ability of polyclonal anti-DARPP-21 antibody to coimmunoprecipitate calmodulin together with DARPP-21 from striatal extracts.

### 6.43 Immobilized Protein-Protein Binding Assays

### 6.4.3.1 Affinity Chromatography using DARPP-21-Sepharose

Purified recombinant DARPP-21 (2 to 5 mg) was covalently crosslinked to 2 ml of Aminolink Sepharose (Pierce Inc., Rockford, Ill.). Frozen striatal extract (2 g) was homogenized in buffer A as previously described. Striatal extract (1 mg/ml) was put through the mock column containing 4 ml Sepharose 4B to avoid potential non-specific protein binding to the beads. The eluate was split into two halves: the first half was applied to the column with immobilized DARPP-21 while the second half was put through the mock column. Columns were washed with 150 mM NaCl and 20 mM Tris pH 7.5, eluted with 10 mM EGTA/20 mM Tris pH 7.5; 2 mM NaCl and 6 M guanidinium HCl. Samples from each elution step were separated by SDS-PAGE, transferred to PVDF membranes and probed with anti-DARPP-21 specific antibody.

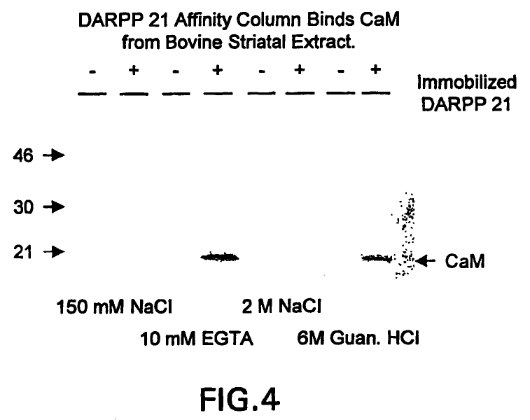

Results indicated that calmodulin was cluted only from the DARPP-21-containing column, not from the mock column. (FIG. 4).

### 6.4.3.2 Affinity Chromatography using Calmodulin-Sepharose

DARPP-21 (0.5 ml of 0.1 mg/ml) was applied to 0.5 ml of 1 mg/ml calmodulin-Sepharose (Amersham Pharmacia Biotech, Piscataway, N.J.) in buffer B (100 mM NaCl, 20 mM Tris pH 7.5, 1 mM MgCl₂, 0.5 mM CaCl₂, 1 mM DTT) or buffer C containing 1 mM EGTA instead of 0.5 mM CaCl₂. Beads were washed with several volumes of buffer D containing 300 mM NaCl and 20 mM Tris pH 7.5 and bound proteins were eluted with 10 mM EGTA/20 mM Tris pH 7.5.

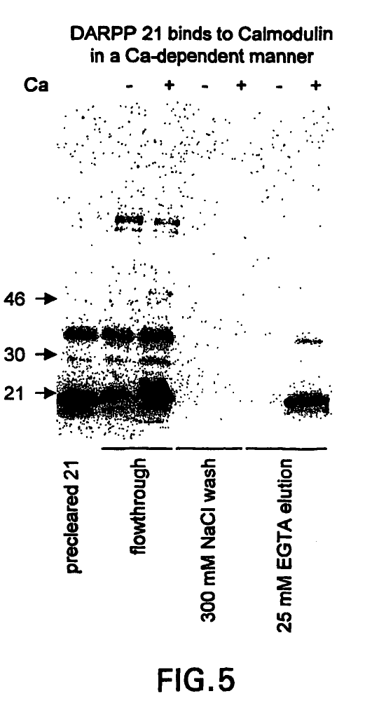

Results indicated that DARPP-21 binding was detected only in the calcium-equilibrated column, and thus the protein-protein interaction was shown to occur in a calcium-dependent manner. (FIG. 5).

### 6.5 Example 5

### Inhibition Of Calcium/calmodulin-Dependent Proteins By Phosphorylated DARPP-21

DARPP-21 was phosphorylated as described in Section 6.1. In vitro inhibition studies on the kinase and phosphatase activities of CaMKI and calcineurin, respectively, were performed according to standard techniques in the art.

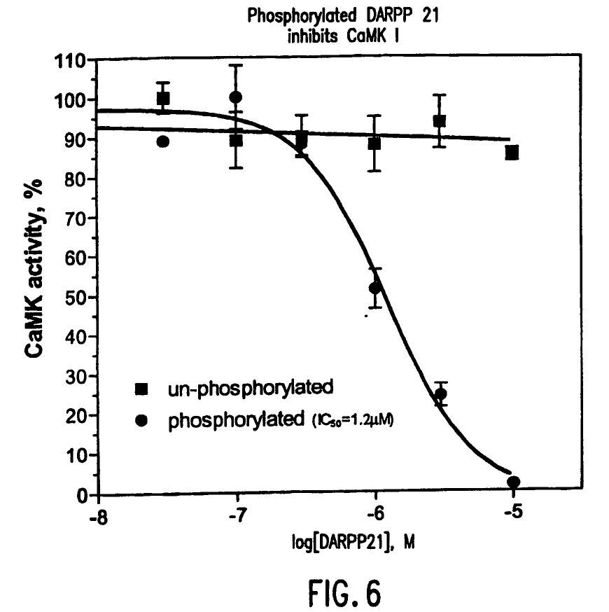

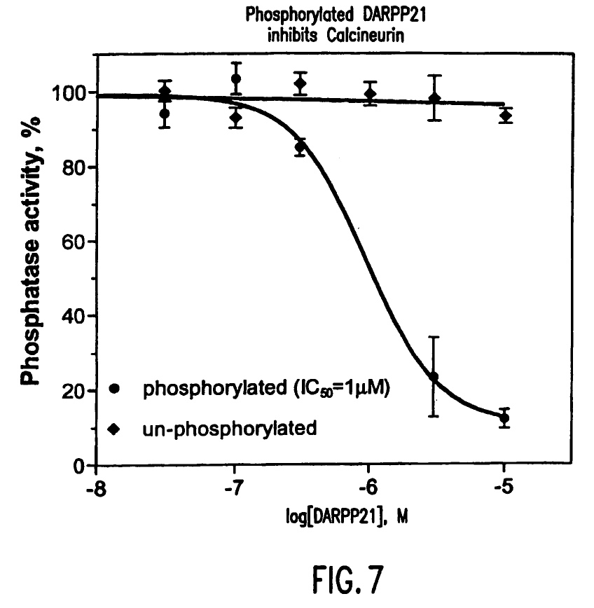

Phosphorylated DARPP-21 was shown to inhibit CaMKI with an IC₅₀ of 1.2 μM. (FIG. 6) Phosphorylated DARPP-21 also inhibited calcineurin with an IC₅₀ of 1 μM. (FIG. 7). The same concentration of unphosphorylated DARPP-21 had no effect on either CaMKI or calcineurin activity. These data indicated that phosphorylated DARPP-21 inhibited both kinase and phosphatase activity through a competitive calcium-dependent binding with calmodulin.

1. A method for modulating a biological activity of DARPP-21 in a cell or tissue, comprising contacting a cell or tissue with an agent that modulates a biological activity of DARPP-21, such that the signaling pathway is modulated.

2. The method of claim 1, wherein the agent alters the phosphorylation state of DARPP-21.

3. The method of claim 1, wherein the agent alters the formation of DARPP-21/calmodulin complexes.

4-7. (canceled)

8. A method for screening agents for activity to modulate calcium signaling in cells or tissue comprising:
(a) contacting a sample of cells or tissue with an agent to be tested for the ability to regulate calcium signaling;
(b) determining the level of phosphorylation of DARPP-21 in the cells or tissue of (a); and
(c) comparing the level determined in (b) to that of a control sample of cells or tissue that were not contacted with the agent, wherein a difference in levels is indicative of the agent to modulate DARPP-21 phosphorylation and thus modulate calcium signaling.

9. The method of claim 8 wherein said cells or tissues are brain cells or tissue.

10. The method of claim 8, further comprising testing the identified agent for an ability to modulate a biological activity of calmodulin-dependent protein kinase I (CaMKI) or protein phosphatase 2B (calcineurin).

11. A method for identifying an agent to be tested for an ability to modulate calcium signaling in cells or tissue, comprising:
(a) contacting DARPP-21 with an agent for a time sufficient to allow binding to DARPP-21;
(b) washing to remove unbound agent; and
(c) assaying for the presence of agent bound to DARPP-21,
so that if the agent binds DARPP-21 then an agent to be tested for an ability to modulate calcium signaling is identified.

12. The method of claim 11, further comprising testing the identified agent for an ability to modulate a biological activity of DARPP-21, calmodulin-dependent protein kinase I (CaMKI) or protein phosphatase 2B (calcineurin).

13. (canceled)

14. A method of screening agents for activity to modulate intracellular calcium signaling comprising:
(a) performing a protein-protein binding assay which includes DARPP-21 and calmodulin in the presence of the agent;
(b) determining the level of binding of DARPP-21 and calmodulin in step (a); and
(c) comparing the level in (b) to the level of binding of a control protein-protein binding assay performed in the absence of the agent, wherein a difference in binding levels is indicative of the agent to modulate intracellular calcium signaling.

15. The method of claim 14, further comprising testing the identified agent for an ability to modulate a biological activity of calmodulin-dependent protein kinase I (CaMKI) or protein phosphatase 2B (calcineurin).

16. A method of screening candidate DARPP-21 binding proteins whose binding with DARPP-21 are inhibited or augmented by calmodulin comprising:
(a) performing a protein-protein binding assay which includes DARPP-21 and a candidate protein in the absence of calmodulin;
(b) performing a protein-protein binding assay which includes DARPP-21 and the candidate polypeptide in the presence of calmodulin;
(c) determining whether calmodulin inhibits or augments the protein-protein binding by comparing the binding of DARPP-21 and the candidate polypeptide in step (a) to that in step (b).

17. A method of screening candidate calmodulin-binding proteins whose binding with calmodulin are inhibited or augmented by DARPP-21 comprising:
(a) performing a protein-protein binding assay which includes calmodulin and a candidate protein in the absence of DARPP-21;
(b) performing a protein-protein binding assay which includes calmodulin and the candidate polypeptide in the presence of DARPP-21;
(c) determining whether DARPP-21 inhibits or augments the protein-protein binding by comparing the binding of calmodulin and the candidate polypeptide in step (a) to that in step(b).

18. The method of claim 17, further comprising testing the identified agent for an ability to modulate a biological activity of calmodulin-dependent protein kinase I (CaMKI) or protein phosphatase 2B (calcineurin).

19. The method of claim 16, wherein DARPP-21 comprises a negative charge at amino acid position 55.

20. The method of claim 16, wherein DARPP-21 is not phosphorylated.

21. The method of claim 16, wherein DARPP-21 is phosphorylated.

22. The method of claim 16, wherein the protein-protein binding assay comprises a two-hybrid assay in yeast or an E. coli/BCCP interactive system.

23. The method of claim 16, wherein the protein-protein binding assay comprises a coimmunoprecipitation or immobilized protein-protein binding assay.

24-28. (canceled)

29. The method of claim 17 wherein DARPP-21 comprises a negative charge at amino acid position 55.

30. The method of claim 17 wherein DARPP-21 is not phosphorylated.

31. The method of claim 17 wherein DARPP-21 is phosphorylated.

32. The method of claim 17 wherein the protein-protein binding assay comprises a two-hybrid assay in yeast or an E. coli/BCCP interactive system.

33. The method of claim 17 wherein the protein-protein binding assay comprises a coimmunoprecipitation or immobilized protein-protein binding assay.
